(19) United States
(12) Patent Application Publication
(10) Pub. No.: US 2023/0133326 A1
(43) Pub. Date: May 4, 2023

(54) STABLE PHARMACEUTICAL COMPOSITIONS OF METFORMIN WITH CONTROL ON NITROSO IMPURITIES

(71) Applicant: The Texas A&M University System, College Station, TX (US)

(72) Inventors: Mansoor Khan, College Station, TX (US); Ziyaur Rahman, College Station, TX (US); Indra K. Reddy, Corpus Christi, TX (US); Satish Dharani, College Station, TX (US)

(21) Appl. No.: 17/975,595

(22) Filed: Oct. 27, 2022

Related U.S. Application Data
(60) Provisional application No. 63/273,134, filed on Oct. 28, 2021.

(51) Int. Cl. A61K 31/155; A61K 31/14; A61K 47/69; A61K 47/34; A61K 33/06; A61K 47/10; A61K 9/16

(52) U.S. Cl. CPC A61K 31/155; A61K 31/14; A61K 47/6951; A61K 47/34; A61K 33/06; A61K 47/10; A61K 9/1617

(57) ABSTRACT

In an embodiment, the present disclosure pertains to a stable formulation of a primary, secondary, and/or tertiary amino group containing composition that prevents or reduces formation of a nitroso impurity until the end of stated expiration or longer. In some embodiments, the composition includes metformin and at least one pharmaceutical excipient. In some embodiments, the at least one pharmaceutical excipient includes a magnesium salt and at least one of a calcium salt, dicalcium phosphate, polyethylene glycol, or polyethylene oxide. In some embodiments, the magnesium salt can include, without limitation, magnesium aluminum silicate, magnesium aluminometasilicate, magnesium carbonate, magnesium oxide, magnesium silicate, magnesium stearate, magnesium sulfate, magnesium trisilicate, and combinations thereof. In some embodiments, a weight ratio of the drug to the at least one pharmaceutical excipient ranges from 1 to 99 to 99 to 1% w/w. In some embodiments, the at least one pharmaceutical excipient is a protective pharmaceutical ingredient.

### CROSS-REFERENCE TO RELATED APPLICATIONS

This patent application claims priority from, and incorporates by reference the entire disclosure of, U.S. Provisional Application 63/273,134 filed on Oct. 28, 2021.

### TECHNICAL FIELD

The present disclosure relates generally to metformin and similar drugs that are known to form nitroso impurities and more particularly, but not by way of limitation, to compositions of stable metformin and similar drug products with control on nitroso impurities.

### BACKGROUND

This section provides background information to facilitate a better understanding of the various aspects of the disclosure. It should be understood that the statements in this section of this document are to be read in this light, and not as admissions of prior art.

Currently, various pharmaceuticals such as metformin (N,N-dimethylimidodicarbonimidic diamide) form nitroso group impurities, including N-nitrosodimethylamine (NDMA). NDMA is a known carcinogen, causing metformin and other drugs to be recalled in several countries. Currently, the Food and Drug Administration (FDA) allows up to 96 ng/day of NDMA in pharmaceuticals. However, if the amount is greater than that, the products are recalled as they are considered harmful. Currently, many pharmaceuticals such as metformin suffer from the formation of NDMA greater than the allowed limit, thus initiating the recall of the products by the FDA.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a stable formulation of a primary, secondary, and/or tertiary amino group containing composition that prevents or reduces formation of a nitroso impurity until the end of stated expiration or longer. In some embodiments, the composition includes metformin and at least one pharmaceutical excipient. In some embodiments, the at least one pharmaceutical excipient includes a magnesium salt and at least one of calcium carbonate, dicalcium phosphate, tricalcium phosphate, calcium sulfate, calcium silicate, calcium trisilicate, calcium stearate, sodium calcium aluminosilicate, polyethylene glycol, or polyethylene oxide. In some embodiments, the magnesium salt can include, without limitation, magnesium aluminum silicate, magnesium aluminometasilicate, magnesium carbonate, magnesium oxide, magnesium silicate, magnesium stearate, magnesium sulfate, magnesium trisilicate, and combinations thereof. In some embodiments, a weight ratio of the drug to the at least one pharmaceutical excipient ranges from 1 to 99 to 99 to 1% w/w. In some embodiments, the at least one pharmaceutical excipient is a protective pharmaceutical ingredient.

In some embodiments, the composition further includes at least one other drug. In some embodiments, the at least one other drug includes a tertiary or quaternary amine or non-amine groups in its structure. In some embodiments, the at least one other drug can include, without limitation, dapagliflozin, empagliflozin, ertugliflozin, and canagliflozin.

In some embodiments, the nitroso impurity can include, without limitation, N-nitrosovarenicline, N-nitrosoquinapril, N-nitroso sitagliptin, N-nitrosodimethylamine, N-nitrosodiethylamine, N-nitroso-N-methyl-4-aminobutanoic acid, N-nitrosoisopropylethyl amine, N-nitrosodiisopropylamine, N-nitrosodibutylamine, N-nitroso-irbesartan, 1-methyl-4-nitrosopiperazine, N-nitrosoquinapril, 1-cyclopentyl-4-nitrosopiperazine and N-nitrosomethylphenylamine as nitroso impurities, nitroso group containing molecules, and combinations thereof. In some embodiments, the composition is stable against formation of the nitroso impurity when the composition packed in high-density polyethylene (HDPE) bottle, blister pack or any of other finished container exposed to 25° C./60% relative humidity (RH) for 18 months or longer or 40° C./75% RH for six months or longer. In some embodiments, the composition reduces formation of the nitroso impurity to an acceptable level. In some embodiments, the acceptable level of nitroso impurity is at or below 26.5 to 96 ng throughout the shelf-life. In some embodiments, the composition is stable against formation of the nitroso impurity, or reduces formation of the nitroso impurity, when the composition is exposed to 25° C./60% relative humidity (RH) or 40° C./75% RH, or during in-use condition (30° C./75% RH).

In some embodiments, the composition has a form including, without limitation, immediate release, extended release, delayed release, delayed extended release, sustained release, controlled release, a tablet, a capsule, a pill, a granule, a pellet, a solution, a suspension, an emulsion, a semi-solid, and combinations thereof. In some embodiments, the at least one pharmaceutical excipient includes a cyclodextrin compound present in the composition in a range from 1 to 95% w/w. In some embodiments, the cyclodextrin compound can include, without limitation, alpha-cyclodextrin, beta-cyclodextrin, gamma-cyclodextrin, randomly methylated beta-cyclodxetrin, hydroxypropyl beta-cyclodextrin, hydroxypropyl gamma-cyclodextrin, sulfobutyl ether beta-cyclodextrin, and combinations thereof.

In some embodiments, the at least one pharmaceutical excipient includes a polydimethylsiloxane compound present in the composition in a range from 1 to 95% w/w. In some embodiments, the polydimethylsiloxane compound can include, without limitation, dimethicone, cyclomethicone, silica dimethyl silylate, simethicone, and combinations thereof. In some embodiments, the at least one pharmaceutical excipient includes a polyhydric alcohol including, without limitation, glycerin, propylene glycol, butylene glycol, propylene carbonate, monothioglycerol, polyethylene glycol (molecular weight of less than 1,000), and combinations thereof. In some embodiments, the polyhydric alcohol is present in the composition in a range from 1 to 95% w/w.

In some embodiments, the at least one pharmaceutical excipient includes a calcium salt present in the composition in a range from 1 to 95% w/w. In some embodiments, the calcium salt can include, without limitation, calcium carbonate, dicalcium phosphate, tricalcium phosphate, calcium sulfate, calcium citrate, calcium pyrophosphate, calcium silicate, calcium trisilicate, calcium stearate, sodium calcium aluminosilicate, and combinations thereof. In some embodiments, the magnesium salt is present in the composition in a range from 1 to 95% w/w.

In some embodiments, the at least one pharmaceutical excipient includes at least one of a sodium, potassium, or aluminum salt present in the composition in a range from 1 to 95% w/w. In some embodiments, the at least one of a of sodium, potassium, and aluminum salt can include, without limitation, sodium silicate, potassium silicate, sodium aluminosilicate, and combinations thereof. In some embodiments, the at least one pharmaceutical excipient includes at least one of kaolin, bentonite, or silicon dioxide present in the composition in a range from 1 to 95% w/w.

In some embodiments, the at least one pharmaceutical excipient includes a long carbon chain acid, saturated or unsaturated, with carbon length varied from 4 to 26. In some embodiments, the long carbon chain acid can include, without limitation, lauric acid, myristic acids, palmitic acids, stearic acid, adipic acid, lipoic acid, omega-3 fatty acids, and combinations thereof. In some embodiments, the at least one pharmaceutical excipient includes a long chain carbon alcohol present in the composition in a range from 1 to 95% w/w. In some embodiments, the long chain carbon alcohol can include, without limitation, cetyl alcohol, cetostearyl alcohol, stearyl alcohol, tocopherol, isobutyl alcohol, myristyl alcohol, octyldodecanol, oleyl alcohol, lanolin alcohols, cholesterol, and combinations thereof. In some embodiments, the at least one of dicalcium phosphate, polyethylene glycol, or polyethylene oxide includes at least one of polyethylene glycol or polyethylene oxide with a molecular weight that varies from 1,000 to 10,000,000.

In some embodiments, the at least one pharmaceutical excipient includes at least one of cellulose acetate, cellulate butyrate, ethyl cellulose, or cellulose acetate present in the composition in a range from 1 to 95% w/w. In some embodiments, the at least one pharmaceutical excipient includes at least one of polydecene or hydrogenated polydecene present in the composition in a range from 1 to 95% w/w. In some embodiments, the pharmaceutical excipient includes a polymer including, without limitation, acrylic acid, amino methacrylate copolymer, ammonio methacrylate copolymer, ethyl acrylate and methyl methacrylate copolymer, methacrylic acid and ethyl acrylate copolymer, methacrylic acid and methyl methacrylate copolymer, and combinations thereof. In some embodiments, the polymer is present in the composition in a range from 1 to 95% w/w.

In some embodiments, the composition includes an antioxidant. In some embodiments, the antioxidant includes, without limitation, butylated hydroxy anisole, butylated hydroxy toluene, sodium/potassium metabisulfites, sodium/potassium sulfite, cysteine, methionine, sodium or calcium ascorbate, fatty acid esters of ascorbic acid, tocopherols, alpha, gamma or delta tocopherol and its esters, 4-hydroxyresorcinol, erythorbic acid, sodium erythorbate, propyl gallate, octyl gallate, tertiary butyl hydroquinone, and combinations thereof. In some embodiments, the antioxidant is present in the composition in a range from 0.001 to 5% w/w. In some embodiments, the composition includes a chelating agent. In some embodiments, the chelating agent includes, without limitation, ethylenediaminetetraacetic acid, edetate sodium, edetate disodium, edetate calcium disodium, edetate tripotassium, edetate dipotassium, and combination thereof. In some embodiments, the chelating agent is present in the composition in a range from 0.001 to 5% w/w. In some embodiments, the magnesium salt includes magnesium stearate. In some embodiments, the at least one of dicalcium phosphate, polyethylene glycol or polyethylene oxide includes polyethylene oxide.

In an additional embodiment, the present disclosure pertains to a stable formulation of a primary, secondary, and/or tertiary amino group containing composition that prevents or reduces formation of a nitroso impurity during its shelf-life. In some embodiments the composition includes metformin and at least one pharmaceutical excipient. In some embodiments, the at least one pharmaceutical excipient includes magnesium stearate and at least one of dicalcium phosphate, polyethylene glycol, or polyethylene oxide. In some embodiments, a weight ratio of the metformin to the at least one pharmaceutical excipient ranges from 1 to 99 to 99 to 1% w/w. In some embodiments, the at least one pharmaceutical excipient is a protective pharmaceutical ingredient.

In another embodiment, the present disclosure pertains to a method of making a stabilized formulation of a primary, secondary, and/or tertiary amino group containing composition that prevents or reduces formation of a nitroso impurity. In general, the method includes adding a pharmaceutical excipient to metformin. In some embodiments, the pharmaceutical excipient includes a magnesium salt and at least one of dicalcium phosphate, polyethylene glycol, or polyethylene oxide. In some embodiments, the magnesium salt can include, without limitation, magnesium aluminum silicate, magnesium aluminometasilicate, magnesium carbonate, magnesium oxide, magnesium silicate, magnesium stearate, magnesium sulfate, magnesium trisilicate, and combinations thereof. In some embodiments, a weight ratio of the metformin to the pharmaceutical excipient ranges from 1 to 99 to 99 to 1% w/w. In some embodiments, the pharmaceutical excipient is a protective pharmaceutical agent.

In some embodiments, the magnesium salt includes magnesium stearate. In some embodiments, the at least one of dicalcium phosphate, polyethylene glycol, or polyethylene oxide includes polyethylene oxide.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates a nitrosamine compound structure.

FIG. 2 illustrates a proposed mechanism for N-nitrosodimethylamine (NDMA) formation.

FIG. 3 illustrates comparative dissolution profiles of commercial and stable extended release (ER) formulations of metformin.

FIG. 4 illustrates comparative dissolution profiles of commercial and stable immediate release (IR) formulations of metformin.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

The U.S. Food and Drug Administration (FDA) has identified N-nitroso-varenicline (NVC), N-nitrosoquinapril, N-nitroso sitagliptin, N-nitrosodimethylamine (NDMA), N-nitrosodiethylamine (NDEA), N-nitroso-N-methyl-4-aminobutanoic acid (NMBA), N-nitrosoisopropylethyl amine (NIPEA), N-nitrosodiisopropylamine (NDIPA), N-nitrosodibutylamine (NDBA), N-nitroso-irbesartan, 1-methyl-4-nitrosopiperazine (MNP), N-nitrosoquinapril, 1-cyclopentyl-4-nitrosopiperazine (CPNP) and N-nitrosomethylphenylamine (NMPA) as nitroso impurities. Nitrosamine compounds are potent genotoxic agents in several animal species, and some are classified as probable or possible human carcinogens by the International Agency for Research on Cancer (IARC). A nitrosamine compound is illustrated in FIG. 1. These compounds have been referred to as “cohort of concern” compounds in the International Council for Harmonization of Technical Requirements for Pharmaceuticals for Human Use (ICH) guidance for industry M7(R1) Assessment and Control of DNA Reactive (Mutagenic) Impurities in Pharmaceuticals to Limit Potential Carcinogenic Risk. The guidance recommends control of any known mutagenic carcinogen, such as, for example, nitroso-compounds, at or below a level such that there would be a negligible human cancer risk associated with the exposure to potentially mutagenic impurities.

Numerous amines containing products with a variety of compositions and methods have been approved by the FDA for marketing and clinical use. The FDA has been recalling angiotensin receptor blockers class of drug (losartan, valsartan, etc.), ranitidine, nizatidine, metformin (N,N-dimethylimidodicarbonimidic diamide), and varenicline commercial formulations due to presence of nitroso impurities. The FDA has recommended acceptable limits of nitroso impurities. Acceptable daily intake limit is 96 ng/day for NDMA and NMBA, 26.5 ng/day for NDEA, NMPA, NIPEA, and NDIPA, and 37 ng/day for NVC. The scale of recalls for a number of commonly used drug products with NDMA impurities are massive and surprising. As an example, a recent list from the FDA indicated 258 entries of recalled products of metformin.

A variety of reasons have been assigned to the formation, or possibility of formation, of NDMA. The FDA guidance on nitroso impurities lists a number of sources of secondary, tertiary, or quaternary amines that can form nitrosamines. These can include, for example, vendor-sourced raw materials, recovered solvents, catalysts, and reagents as sources of contamination, quenching processes in certain reaction mixtures, and a lack of process optimization and/or control.

Furthermore, NDMA amounts were found to be different for the same product in different lots. The FDA has also indicated that the NDMA impurities have only been observed in extended release products of metformin hydrochloride, but not in immediate release products. However, as disclosed herein, tests have indicated surprising results of NDMA impurities in the immediate release products as well. Studies have indicated that manufacturing processes and sources of water used, not the excipients, are responsible for NDMA impurities. However, as discussed in further detail below, the findings of the present disclosure indicated that certain excipients are, indeed responsible for NDMA formation.

Metformin hydrochloride is a highly prescribed drug product, currently marketed as brand and generic drug products from several companies. It is the first line treatment for type 2 diabetes, with over 150 million prescriptions written per year worldwide, and over 80 million in the Unites States alone. Currently, marketed formulations have shown surprisingly high and unacceptable levels of NDMA impurity to which several products have needed to be recalled from the market. Owing to the unexpected development of the stated carcinogenic impurity, companies are having to recall their product. They are preparing fresh lots and reintroducing the products into the market. However, they will undoubtedly fail again because the underlying reasons are not corrected.

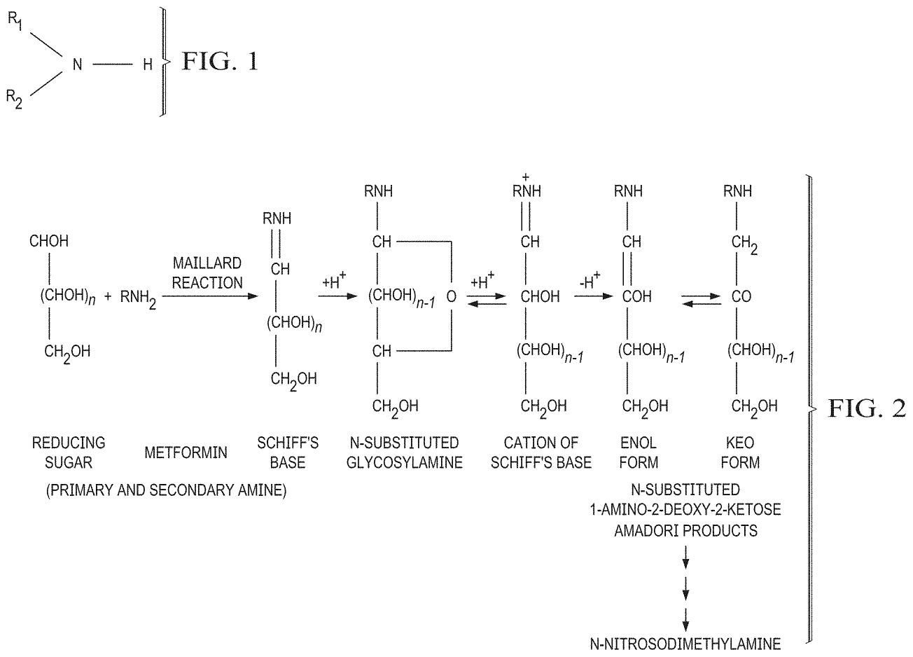

It has been surprisingly discovered that several FDA approved metformin products are repeatedly failing, and thus getting recalled from the market, due to the formation of NDMA impurities. FIG. 2 illustrates a proposed mechanism for NDMA formation. Several excipients can cause a reaction with metformin or similar products to form nitroso impurities via the pathway shown in FIG. 2. Some of these are potentially present in commercial metformin products. A list of excipients in commercial products include, for example, crospovidone, hypromellose 2208 (100 mPa·s), hypromellose 2208 (100,000 mPa·s), hypromellose 2208 (15,000 mPa·s), hypromellose 2910 (6 mPa·s), povidone K30, povidone K90, sodium carboxymethyl cellulose (CMC), microcrystalline cellulose, sodium starch glycolate, lactose monohydrate, stearic acid, oleic acid, ethylcelluloses, ammonia, polyethylene glycol 400, medium-chain triglycerides, povidone, titanium dioxide, triacetin, xanthan gum, sodium carbonate, talc, hypromellose, ammonio methacrylate copolymer type A, ammonio methacrylate copolymer type B, silicon dioxide, dibutyl sebacate, magnesium stearate, and microcrystalline cellulose (MCC). Sampling a few recalled metformin products (e.g., 500 mg extended release and 500 or 750 mg immediate release formulations) with common excipients, such as hypromelloses, povidones, lactose, and MCC were found in several FDA approved metformin products.

Laboratory efforts, as discussed in further detail herein, have shown that metformin stable dosage forms of primary, secondary, and/or tertiary amino groups containing drugs can be formulated with protective pharmaceutical excipients. The stable formulation prevents or reduces the formation of nitrosamine impurities on exposure to room temperature (25° C./60% relative humidity; RH), in-use (30° C./75% RH), and at accelerated temperature and/or humidity conditions (40° C./75% RH) for the shelf life or estimated shelf life of the product ranging from one year to two years or more.

Accordingly, the present disclosure pertains to various pharmaceutical compositions of primary, secondary, and/or tertiary amine group containing drugs which are either stable against the formation of nitroso impurities, or reduce the formation and keep the nitroso impurities level below the FDA recommended level when stored at room temperature (25° C./60% RH), in-use condition (30° C./75% RH), and high temperature and/or humidity conditions (40° C./75% RH).

### Definitions

To facilitate understanding of the present disclosure, several terms are defined herein below. Those left undefined have meanings as commonly understood by a person of ordinary skill in the art relevant to this disclosure. Although various examples are provided with respect to the definitions given below, it will be understood that these examples impose no limitations to the definitions as set forth herein.

The term “similar drugs” describe primary, secondary, and/or tertiary amino group containing drugs that are known/potential to form nitroso impurities.

The term “nitrosamine impurity” describes a class of compounds having the chemical structure of a nitroso group bonded to an amine (R₁N(—R₂)—N═O). Examples of nitrosamine impurities, can include, without limitation, N-nitrosodimethylamine (NDMA), N-nitrosodiethylamine (NDEA), N-nitroso-N-methyl-4-aminobutanoic acid (NMBA), N-nitrosoisopropylethyl amine (NIPEA), N-nitrosodiisopropylamine (NDIPA), N-nitrosodibutylamine (NDBA), N-nitroso-varenicline (NVC), N-nitroso-irbesartan, N-nitroso sitagliptin, 1-methyl-4-nitrosopiperazine (MNP), 1-cyclopentyl-4-nitrosopiperazine (CPNP), and N-nitrosomethylphenylamine (NMPA).

The term “primary, secondary, and/or tertiary amino group containing drugs” are those drugs that contain at least a primary, secondary, or tertiary amine group in their structure. Examples of primary, secondary, and/or tertiary amino group containing drugs, can include, without limitation, metformin, varenicline, quinapril, nizatidine, glipizide, glyburide, glimepiride, pioglitazone, rosiglitazone, repaglinide, alogliptin, linagliptin, sitagliptin, saxagliptin, candesartan, irbesartan, losartan, Olmesartan, valsartan, azilsartan, hydrochlorothiazide, amlodipine, nebivolol, sacubitril, aliskiren, ranitidine, cimetidine, omeprazole, rifampin, rifapentine, and their salts. Any drug that does not contain either a primary, secondary, or tertiary amine group does not belong to this class.

The term “stable formulation”, “stable pharmaceutical composition”, and “stable dosage forms” are used interchangeably. They refer to pharmaceutical compositions packed in a high-density polyethylene (HDPE) bottle, blister packed or amber colored pharmacy vials, and stable against, or reduce the formation of nitrosamine impurities to acceptable limits when exposed to 40 ° C./75% relative humidity (RH) for six months or longer or 25° C./60% RH for 18 months or longer or both from the date of manufacture. Additionally, they refer to pharmaceutical compositions that are stable against, or reduce the formation of nitrosamine impurities to acceptable limits when in-use. For example, such that NDMA impurity is within the acceptable limit of 96 ng/mL throughout the shelf life of at least one year or more at room temperature. When stored in a low-density amber pharmacy vials, it is stable of at least 3 months.

The terms “immediate release” (IR) mean a release of majority of the drug to an aqueous environment over a period of seconds to no more than about 120 minutes.

The terms “extended release” or “extended-release” (ER) assume the definition as widely recognized to those of ordinary skill in the art of pharmaceutical sciences. For example, an extended-release dosage form will release the drug slowly over an extended period (e.g., 4, 6, 8, 10, 12, 16, 20, or 24 hours).

The terms “delayed release” or “enteric release” are used interchangeably. They refer to pharmaceutical compositions that prevents drug release in acidic pH of stomach but released the drug in lower part of duodenum or small intestine.

The terms “delayed extended release” or “enteric extended release” are used interchangeably. They refer to pharmaceutical compositions that prevents drug release in acidic pH of stomach but released the drug in lower part of duodenum or small intestine in extended release manner.

The term “pills” are drug-containing tablets or capsules of all sizes and shapes intended for oral administration in humans.

The term “pellets” are dosage forms composed of small, solid particles of uniform shape sometimes called “beads”. Typically, pellets are nearly spherical, but this is not required. Pellets may be administered orally (gastrointestinal) or by injection.

The term “solution” refers to a homogenous molecular mixture of a pharmaceutical composition where all the composition components are present in molecular form.

The term “suspension” refers to non-homogenous particulate dispersion of pharmaceutical composition in a liquid vehicle. At least one of the components is present in particulate form in the composition.

The term “emulsion” refers to non-homogenous droplet dispersion of a pharmaceutical composition in liquid components. The liquid components are not miscible when mixed together.

The term “semi-solid” refers to a pharmaceutical composition where consistency of the formulation falls in between solid and liquid. Examples of semi-solid dosage forms can include, without limitations, creams, pastes, gels, ointments, lotions, liniments, and the like.

The terms “pharmaceutically acceptable excipient”, “excipient”, and “pharmaceutically acceptable carrier” are used interchangeably. They refer to a substance, other than the primary, secondary, and/or tertiary amino group containing drugs, with which the drug is formulated.

The terms “protective pharmaceutically excipient”, “protective acceptable pharmaceutical excipient”, “protective excipient”, and “protective pharmaceutically acceptable carrier” are used interchangeably. They refer to a substance, other than the primary, secondary, and/or tertiary amino group containing drugs, with which the drug is formulated to protect and/or reduce the formation of nitrosamine impurities. Protective pharmaceutical excipients belong to the following categories that can include, without limitation, cyclodextrin, dimethylsulfoxide, polyhydric alcohols, calcium salts, magnesium salts, sodium silicate, potassium silicate, aluminum silicate, long chain carbon acids, sodium, calcium, magnesium and zinc salts of long chain carbon acids, long chain carbon alcohols, esters of long chain carbon acids, long chain hydrocarbons, polymer of ethylene glycol, ester of cellulose, non-ionic surfactants, volatile oils, esters of sucrose derivatives, acrylate polymers, esters of citrate derivatives, polydecene, and polydecene hydrogenated.

Protective excipients of cyclodextrin are a family of cyclic oligosaccharide and are composed of five or more α-D-glucopyranoside units linked 1→4. Examples of cyclodextrin include, without limitation, alpha-cyclodextrin, beta-cyclodextrin, gamma-cyclodextrin sulfobutyl ether beta-cyclodextrin, hydroxypropyl beta-cyclodextrin, methylated beta-cyclodextrin, and randomly methylated beta-cyclodextrin.

Examples of protective excipients of a dimethylsilane category includes, without limitation, dimethicone, silica dimethyl silylate, simethicone, and cyclomethicone.

Protective excipients of polyhydric alcohols contain at least two alcohol group. Examples of polyhydric alcohol include, without limitation, glycerin, propylene glycol, hexylene glycol, xylitol, sorbitol, propylene carbonate, butylene glycol, polyethylene glycol, and monothioglycerol.

Protective excipients of calcium salts contain calcium and non-calcium parts. Non-calcium parts can be carbon or non-carbon. Examples of calcium salts include, without limitation, dicalcium phosphate, tricalcium phosphate, calcium carbonate, calcium sulfate, calcium stearate, calcium citrate, calcium pyrophosphate, calcium silicate, sodium calcium aluminosilicate, and tricalcium silicate.

Protective excipients of magnesium salts contain magnesium and non-magnesium parts. Non-magnesium parts can be carbon or non-carbon. Examples of magnesium salts include, without limitation, magnesium carbonate, magnesium oxide, magnesium sulfate, magnesium stearate, magnesium silicate, magnesium trisilicate, magnesium aluminum silicate, talc, magnesium aluminometasilicate, and attapulgite.

Protective excipients of sodium, potassium, and/or aluminum are silicate minerals. Examples of sodium, potassium, and/or aluminum silicate include, without limitation, sodium silicate, potassium silicate, and sodium aluminosilicate.

Protective excipients of long carbon chain acids category contain saturated and unsaturated 4-26 carbons. Examples include, without limitation, stearic acid, lauric acid, myristic acid, palmitic acid, oleic acid, lauric acid, caprylic acid, adipic acid, tocopherol, lipoic acid, omega-3-fatty acids, and sorbic acid.

Protective excipients of sodium, potassium, magnesium, zinc, and aluminum salt of long chain carbon acids contain 4-26 carbons, saturated and/or unsaturated. Examples of sodium, potassium, magnesium, zinc, and aluminum salt of long chain carbon acids include, without limitation, sodium stearate, sodium lauryl sulfate, calcium stearate, magnesium stearate, aluminum monostearate, sodium stearyl fumarate, zinc stearate, and sodium cetostearyl sulfate.

Protective excipients of long carbon chain alcohols contain 4-26 carbons, saturated and/or unsaturated. Examples of long carbon chain alcohols include, without limitation, cetyl alcohol, cetostearyl alcohol, cholesterol, stearyl alcohol, isobutyl alcohol, myristyl alcohol, octyldodecanol, oleyl alcohol, lanolin alcohols, and inositol.

Protective excipients of esters of long carbon chain category contains saturated and/or unsaturated acids. Examples of ester of long carbon chain esters include, without limitation, almond oil, peanut oil, sesame oil, soybean oil, corn oil, cottonseed oil, coconut oil, coconut oil, hydrogenated, palm kernel oil, palm oil, palm oil, hydrogenated, rapeseed oil, fully hydrogenated, rapeseed oil, superglycerinated fully hydrogenated, sunflower oil, cetyl palmitate, canola oil, castor oil, safflower oil, soybean oil, hydrogenated, castor oil, hydrogenated, vegetable oil, hydrogenated, type I, olive oil, diacetylated monoglycerides, ethyl oleate, hard fat, cocoa butter, glyceryl behenate, glyceryl dibehenate, ethylene glycol stearates, glyceryl monooleate, glyceryl monostearate, isopropyl isostearate, isopropyl myristate, isopropyl palmitate, isostearyl isostearate, oleyl oleate, glyceryl distearate, glyceryl mono and dicaprylate, glyceryl mono and dicaprylocaprate, glyceryl monocaprylate, glyceryl monocaprylocaprate, glyceryl monolinoleate, glyceryl tricaprylate, glyceryl tristearate, medium-chain triglycerides, triacetin, and mono- and di-glycerides.

Protective excipients of esters of long carbon chain category contain greater than 10-30 carbons saturated and/or unsaturated acids. Examples include, without limitation, paraffin, mineral oil, petrolatum, hydrogenated lanolin, wax, microcrystalline, wax, carnauba, bee wax, and candelilla wax.

Protective excipients of polyethylene glycol contain polymers of ethylene glycol having 100-10,000,000 molecular weight. Examples of polymer of ethylene glycol are polyethylene glycol and polyethylene oxide.

Protective excipients of cellulose ester category are acetate, ethyl, and butyl esters of cellulose. Examples include, without limitation, cellulose acetate, cellulose acetate butyrate, and ethyl cellulose.

Protective excipients of non-ionic surfactants contain polar head groups that are not electrically charged. Examples of non-ionic surfactants include, without limitation, diethylene glycol monoethyl ether, egg phospholipids, propylene glycol monolaurate, propylene glycol dilaurate, polypropylene glycol 11 stearyl ether, lauroyl polyoxylglycerides, linoleoyl polyoxylglycerides, polyglyceryl 3 diisostearate, polyglyceryl dioleate, polyoxyl 10 oleyl ether, polyoxyl 15 hydroxystearate, polyoxyl 20 cetostearyl ether, polyoxyl 35 castor oil, polyoxyl 40 castor oil, hydrogenated, polyoxyl 40 stearate, polyoxyl lauryl ether, polyoxyl stearate, tyloxapol, polyoxyl stearyl ether, polysorbate 20, polysorbate 40, polysorbate 60, polysorbate 80, sorbitan monolaurate, sorbitan monooleate, sorbitan monopalmitate, sorbitan monostearate, sorbitan sesquioleate, sorbitan trioleate, stearoyl polyoxylglycerides, caprylocaproyl polyoxylglycerides, oleoyl polyoxylglycerides, nonoxynol 9, octoxynol 9, vitamin E polyethylene glycol succinate, polyethylene glycol monomethyl ether, and polaxamers.

Protective excipients of volatile oils contain either volatile components and/or components that volatize at room temperature. Example of volatile oils include, without limitation, menthol, peppermint, peppermint oil, peppermint spirit, rose oil, thymol, anise oil, and eucalyptus oil.

Protective excipients of sucrose derivatives contain esters between sucrose and fatty acids of 4-26 carbons. Examples of sucrose derivatives include, without limitation, sucrose diacetate hexaisobutyrate, sucrose palmitate, and sucrose stearate.

Protective excipients of acrylate polymers are copolymer of amino methacrylate, ammonio methacrylate, acrylic acid (CARBOPOL®), ethyl acrylate, methyl methacrylate, methacrylic acid ethylene glycol, and/or vinyl alcohol. Examples of acrylate polymers include, without limitation, amino methacrylate copolymer, ammonio methacrylate copolymer, ethyl acrylate and methyl methacrylate copolymer, ethylene glycol and vinyl alcohol graft copolymer, methacrylic acid and ethyl acrylate copolymer, and methacrylic acid and methyl methacrylate copolymer.

Protective excipients of derivatives of citrate are esters of citric acids. Examples of derivatives of citrates include, without limitation, acetyltributyl citrate, acetyltriethyl citrate, tributyl citrate, and triethyl citrate.

Antioxidants are substances that act as a reductant and reacts with an oxidant, and therefore, prevent oxidation/autooxidation reaction of degradation. Examples of antioxidants are, without limitation, butylated hydroxy anisole (BHA), butylated hydroxy toluene (BHT), sodium/potassium metabisulfites, sodium/potassium sulfite, cysteine, methionine, sodium or calcium ascorbate, fatty acid esters of ascorbic acid, tocopherols, alpha, gamma or delta tocopherol and its esters, 4-hydroxyresorcinol, erythorbic acid, sodium erythorbate, propylgallate, octyl gallate, ascorbic acid, tertiary butyl hydroquinone, and combinations thereof.

Chelating agent are chemical compounds that react with metal ions from participating in oxidation reaction. It forms a stable, water-soluble complex. They are also known as chelants, chelators, or sequestering agents. Examples of chelating agent are, without limitation, ethylenediaminetetraacetic acid, edetate sodium, edetate disodium, edetate calcium disodium, edetate tripotassium, edetate dipotassium, and combination thereof.

Categories of non-protective excipients include, for example, diluents, disintegrants, super-disintegrants, lubricants, glidants, binders, hydrophilic polymers, surfactants, coatings, and the like. Examples of non-protective excipients include, without limitation, carrageenan, hydroxypropyl methylcellulose, hydroxypropyl cellulose, sucralose, xylose, chondroitin sulfate sodium, psyllium, acarbose, acetylated distarch adipate, acetylated distarch oxypropanol, acetylated distarch phosphate, acetylated distarch glycerol, pectinic acid, sorbose, carob bean gum, carrageenan, carboxymethyl cellulose, sodium carboxymethyl cellulose, dextran, methyl acrylate, ethyl acrylate, succinyl distarch glycerol, starch sodium succinate, starch sodium octenyl succinate, starch aluminum octenyl succinate, starch acetate, sodium hydroxide gelatinized starch, hydroxypropyl starch, hydroxypropyl distarch phosphate, hydroxypropyl distarch glycerol, distarch phosphate, chitosan, pectin, pectinic acid, distarch oxypropanol, distarch glycerol, gellan gum, tragacanth, povidone, carrageenan, sodium alginate, sodium starch glycolate, xylitol, alginic acid, croscarmellose sodium, guar gum, sorbitol, starch, pregelatinized starch, hydroxypropyl starch, hydrogenated starch hydrolysate, maltose, lactitol, microcrystalline cellulose, cellulose, dextrates, dextrin, dextrose, erythritol, fructose, invert sugar, sucrose diacetate hexaisobutyrat, caramel, hydroxyethyl cellulose, hypromellose acetate succinate, hypromellose phthalate, isomalt, maltitol, ethylcellulose, ethyl acrylate and methyl methacrylate copolymer, cellaburate, cellacefate, cellulose acetate, carboxymethylcellulose calcium, carboxymethylcellulose sodium, galactose, inositol, gelatin, lactose, mannitol, trehalose, pullulan, polydextrose, and tagatose.

### WORKING EXAMPLES

Reference will now be made to more specific embodiments of the present disclosure and data that provides support for such embodiments. However, it should be noted that the disclosure below is for illustrative purposes only and is not intended to limit the scope of the claimed subject matter in any way.

NDMA impurities can be analyzed by a variety of methods. Any method that is used needs to be validated and one such method is developed and validated as described herein.

Liquid chromatography-mass spectrometry (LCMS) method development for NDMA. Ultra-pressure liquid chromatography (UPLC) was used to detect NDMA in samples. An ACQUITY™ UPLC™ H-Class PLUS System was utilized with the following configuration. The needle was washed with 80:20 methanol:water and the sample temperature was 10° C. The injection volume was 25 μL and a WATERS™ Xselect HSS T3 2.5 μm 2.1×100 mm column was used having a column temperature of 40° C. Mobile phase A was 0.02% formic acid in water, and mobile phase B was acetonitrile. A 0.4 mL/min flow rate was utilized. Table 1 and Table 2, shown below, illustrate gradient method parameters and LCMS method validation for NDMA, respectively.

TABLE 1
Gradient method.

| Time (min) | % A | % B |
|---|---|---|
| 0 | 95 | 5 |
| 3.5 | 95 | 5 |
| 3.51 | 5 | 95 |
| 6 | 5 | 95 |
| 6.01 | 95 | 5 |
| 10 | 95 | 5 |

TABLE 2
LCMS method validation for NDMA.

| | | | | |
|---|---|---|---|---|
| | | | | Mean/Mean ± |
| | | | | Standard Deviation |
| Parameters | Day 1 | Day 2 | Day 3 | (SD) |

Linearity

| | | | | |
|---|---|---|---|---|
| Correlation coefficient | 0.992 | 0.99 | 0.996 | 0.993 ± 0.003 |
| Slope | 2532.3 | 2511.1 | 2634.8 | 2559.4 ± 66.15 |
| y-Intercept | 1183.6 | 1147.613 | 1691.2 | 1340.8 ± 303.98 |
| Analytical range (ng/mL) | | | 10-100 | |
| Calibrators | 8 | 8 | 8 | 8 |
| Limit of Detection | 1.728 | 1.728 | 1.728 | 1.728 |
| (LOD; ng/mL) | | | | |
| Limit of Quantitation | 5.238 | 5.238 | 5.238 | 5.238 |
| (LOQ; ng/mL) | | | | |

Accuracy (% Relative Standard Deviation (RSD), n = 5)

| | | | | |
|---|---|---|---|---|
| 10 ng/mL | 104.05 | 97.069 | 101.229 | 100.78 ± 3.51 |
| 30 ng/mL | 104.93 | 104.52 | 102.388 | 103.94 ± 1.36 |
| 50 ng/mL | 104.38 | 105.62 | 106.83 | 105.61 ± 1.22 |
| 100 ng/mL | 100.91 | 110.34 | 102.94 | 104.73 ± 4.96 |

Precision (% RSD, n = 5)

| | | | | |
|---|---|---|---|---|
| 10 ng/mL | 2.638 | 3.453 | 3.387 | 3.16 ± 0.45 |
| 30 ng/mL | 1.61 | 1.838 | 2.001 | 1.81 ± 0.19 |
| 50 ng/mL | 3.725 | 0.958 | 3.422 | 2.70 ± 1.51 |
| 100 ng/mL | 3.34 | 4.1 | 5.5102 | 4.31 ± 1.1 |

Screening excipients. Excipients were screened to determine which excipients were promoting nitroso impurities. Metformin hydrochloride and excipients were physically mixed in 1:1 weight ratio and exposed to 40° C./75% RH for 1-4 weeks (Table 3).

TABLE 3
Excipients screening for NDMA formation.

| | | NDMA (ng/500 mg) | | |
|---|---|---|---|---|
| Drug | Excipient | Initial | 1-Week | 4-Week |
| Metformin | Hydroxypropyl | 55.0 ± 3.4 | 51.2 ± 8.3 | 49.7 ± 7.3 |
| Hydrochloride | Methylcellulose K4 M | | | |
| | Hydroxypropyl | 18.8 ± 4.0 | 30.5 ± 2.3 | 43.4 ± 5.1 |
| | Methylcellulose K15 M | | | |
| | Hydroxypropyl | 25.9 ± 9.0 | 33.2 ± 3.7 | 46.7 ± 6.8 |
| | Methylcellulose K100 M | | | |
| | Hydroxypropyl Cellulose | 0.0 | 0.0 | 0.0 |
| | Polyvinylpyrrolidone K15 | 17.6 ± 1.7 | 25.2 ± 4.1 | 24.0 ± 9.9 |
| | Polyvinylpyrrolidone K85 | 15.3 ± 3.6 | 31.6 ± 3.1 | 30.6 ± 16.2 |
| | Polyvinyl Alcohol-L | 0.0 | 27.8 ± 2.4 | 33.2 ± 5.6 |
| | Polyvinyl Alcohol-H | 18.4 ± 2.8 | 28.2 ± 1.3 | 42.3 ± 4.1 |
| | Ethylcellulose (4 cps) | 0.0 | 0.0 | 0.0 |
| | Ethylcellulose (20 cps) | 0.0 | 0.0 | 0.0 |
| | Lactose Anhydrous | 0.0 | 22.7 ± 1.6 | 23.6 ± 4.7 |
| | Lactose Monohydrate | 0.0 | 24.1 ± 2.1 | 26.8 ± 3.6 |
| | Microcrystalline Cellulose 101 | 15.9 ± 5.0 | 22.4 ± 2.3 | 22.9 ± 5.2 |
| | Mannitol | 15.3 ± 9.0 | 23.4 ± 1.9 | 25.1 ± 4.5 |
| | Sodium Carboxymethyl Cellulose | 39.9 ± 10.7 | 37.5 ± 1.2 | 57.6 ± 5.5 |
| | Magnesium Stearate | 0.0 | 0.0 | 0.0 |
| | Cellulose Acetate | 0.0 | 0.0 | 0.0 |
| | Cellulose Acetate Butyrate | 0.0 | 0.0 | 0.0 |
| | Sodium Starch Glycolate | 25.0 ± 4.2 | 24.1 ± 1.4 | 26.7 ± 3.2 |
| | Starch | 14.6 ± 7.5 | 28.5 ± 3.7 | 36.8 ± 8.5 |

Table 4, shown below, illustrates screening of additional excipients Samples were exposed to open conditions (40° C./75% RH) for 1 week.

TABLE 4
Excipients screening for NDMA formation.

| | NDMA Impurity (ng/500 mg Drug) | |
|---|---|---|
| Metformin:Excipient (1:1) | Initial | 1-Week |
| CARBOPOL ® 2020NF | 0.0 | 7.9 ± 2.3 |
| Chitosan | 0.0 | 4.0 ± 1.6 |
| CARBOPOL ® 974 | 0.0 | 14.9 ± 5.4 |
| Dicalcium Phosphate | 0.0 | 0.0 |
| D-Sorbitol | 0.0 | 13.6 ± 4.8 |
| EUDRAGIT ® E100 | 333.1 ± 25.6 | 407.3 ± 31.6 |
| EUDRAGIT ® L100 | 0.0 | 4.0 ± 2.6 |
| EUDRAGIT ® RLPO | 0.0 | 0.0 |
| EUDRAGIT ® RSPO | 0.0 | 0.0 |
| EUDRAGIT ® S100 | 0.0 | 3.4 ± 1.5 |
| Gelatin | 0.0 | 0.0 |
| HPMC-AS | 0.0 | 18.2 ± 6.4 |
| KOLLICOAT ® MAE-100-55 | 0.0 | 0.0 |
| KOLLICOAT ® IR | 0.0 | 0.0 |
| KOLLIDON ® VA64 | 0.0 | 0.0 |
| KOLLIDON ® 30 | 0.0 | 0.0 |
| KOLLIDON ® SR | 0.0 | 0.0 |
| KOLLIWAX ® HCO | 0.0 | 10.2 ± 2.8 |
| KOLLIWAX ® SA | 0.0 | 43.5 ± 9.7 |
| MCC-102 | 15.9 ± 5.6 | 22.4 ± 4.7 |
| Polycarbophil | 0.0 | 5.4 ± 1.5 |
| POLYOX ™ | 0.0 | 0.0 |
| Sod Saccharin | 0.0 | 0.0 |
| Sodium Alginate | 0.0 | 15.9 ± 2.8 |
| Xanthan Gum | 0.0 | 0.0 |

Example of control formulations are shown below in Table 5. Briefly, metformin is mixed with polymer(s) followed by lubrication with magnesium stearate and compression. The tablets were exposed to 40° C./75% RH for 2-weeks in a pharmacy vial and monitored for NDMA impurity. These formulations (MF1-MF10) failed to meet FDA limit of NDMA impurity (Table 6).

TABLE 5
Composition of metformin control formulations.

| Ingredient | MF1 | MF2 | MF3 | MF4 | MF5 | MF6 | MF7 | MF8 | MF9 | MF10 |
|---|---|---|---|---|---|---|---|---|---|---|
| Metformin (mg) | 500 | 500 | 500 | 500 | 500 | 500 | 500 | 500 | 500 | 500 |
| Hydroxypropyl | | | | | | | | | | |
| Methylcellulose- | 500 | 0 | 0 | 0 | 0 | 0 | 0 | 250 | 0 | 0 |
| K15 M | | | | | | | | | | |
| Hydroxypropyl | | | | | | | | | | |
| Methylcellulose- | 0 | 500 | 0 | 0 | 0 | 0 | 0 | 0 | 250 | 0 |
| K100 M | | | | | | | | | | |
| Hydroxypropyl | 0 | 0 | 500 | 0 | 0 | 0 | 0 | 0 | 0 | 0 |
| Cellulose | | | | | | | | | | |
| Polyvinylpyrrolid | 0 | 0 | 0 | 500 | 0 | 0 | 0 | 0 | 0 | 250 |
| One-K15 | | | | | | | | | | |
| Polyvinylpyrrolid | 0 | 0 | 0 | 0 | 500 | 0 | 0 | 0 | 0 | 0 |
| One-K85 | | | | | | | | | | |
| Polyvinyl | 0 | 0 | 0 | 0 | 0 | 500 | 0 | 0 | 0 | 0 |
| Alcohol-L | | | | | | | | | | |
| Polyvinyl | 0 | 0 | 0 | 0 | 0 | 0 | 500 | | 0 | 0 |
| Alcohol-H | | | | | | | | | | |
| POLYOX ™ | | | | | | | | | | |
| (WSR 303 LEO | 0 | 0 | 0 | 0 | 0 | 0 | 0 | 250 | 250 | 250 |
| NF) | | | | | | | | | | |
| Magnesium | 10 | 10 | 10 | 10 | 10 | 10 | 10 | 10 | 10 | 10 |
| Stearate | | | | | | | | | | |
| Total (mg) | 1010 | 1010 | 1010 | 1010 | 1010 | 1010 | 1010 | 1010 | 1010 | 1010 |

TABLE 6
Control formulations-NDMA level after
exposure to 40° C./75% RH for 2-weeks.

| | NDMA in ng/500 mg of Metformin Tablet (±SD) | | |
|---|---|---|---|
| Formulation | Initial | 1 W | 2 W |
| MF1 | 0 | 422.4 ± 26.1 | 478.2 ± 28.2 |
| MF2 | 0 | 279.0 ± 19.8 | 325.1 ± 24.3 |
| MF3 | 0 | 316.3 ± 32.4 | 392.5 ± 30.1 |
| MF4 | 0 | 259.0 ± 22.8 | 340.2 ± 36.3 |
| MF5 | 0 | 411.4 ± 50.2 | 487.6 ± 43.8 |
| MF6 | 0 | 494.2 ± 37.2 | 491.0 ± 38.6 |
| MF7 | 0 | 267.1 ± 22.7 | 343.2 ± 34.6 |
| MF8 | 0 | 426.2 ± 29.8 | 448.6 ± 27.6 |
| MF9 | 0 | 310.5 ± 34.8 | 352.8 ± 39.2 |
| MF10 | 0 | 220.6 ± 32.6 | 280.3 ± 38.5 |

Previous studies have been conducted on various formulations of a primary, secondary, and/or tertiary amino group containing composition. These studies utilized, for example, metformin tablet batches that were manufactured, on a batch level, using intra-granular binders blend of polyvinyl pyrrolidone (PVPK30) and hydroxypropyl methyl cellulose (HPMC) E5 or hydroxypropyl methyl cellulose (HPMC) E5 for immediate-release tablets, and blend of carboxymethyl cellulose sodium 4000 (CMC Na) and HPMC K15M or blend of hydroxypropyl cellulose SSL (HPC SSL) and polyethylene oxide (e.g., POLYOX™) were utilized in sustained-release metformin tablets.

These tablet formulations contained blend of HPMC E5 and PVP K30 or CMC Na 4000 and HPMC K15M or HPC SSL and POLYOX™ as rate controlling polymers. These studies, however, did not evaluate the tablet formulation for stability. As shown above, metformin tablets using these various polymers, and similar, were prepared and evaluated.

These tablets showed zero NDMA level at initial times, but eventually failed in stability testing (Table 5 and Table 6). In fact, most of the commercially available products contain HPMC, HPC and CMC polymers, and these products failed to meet NDMA limits upon storage. The formulations, as discussed below, of the present disclosure are stable against, and reduce generation of NDMA when exposed to 40° C./75% RH in a pharmacy vial for a year.

Additionally, studies on various formulations were prepared as sustained release tablets using blend of HPC SSL and polyethylene oxide (e.g., POLYOX™) by wet granulation methods, and evaluated for NDMA levels at initial time points only. Again, these studies did not evaluate the tablet formulation for stability. Tablet formulation of metformin using HPC or blend of HPC SSL and polyethylene oxide did not prevent formulation of NDMA impurity when exposed to 40° C./75% RH for 1 week.

Table 7, shown below, illustrates lab evaluation of NDMA in 500 mg commercial metformin tablets stored at in-use stability condition (30° C./75% RH) for 12 weeks. Product types include immediate release (IR) and extended release (ER).

TABLE 7
Lab evaluation of NDMA in 500 mg commercial metformin tablets.

| | | NDMA in ng/500 mg of Metformin Tablet (±SD) | | | | |
|---|---|---|---|---|---|---|
| Product | Type | Initial | 2 W-In Use | 4 W-In Use | 8 W-In Use | 12 W-In Use |
| M1 | IR | 0 | 0 | 0 | 0 | 0 |
| M2 | IR | 1164.0 ± 52.9 | 1824.1 ± 83.0 | 2108.6 ± 500.2 | 2882.5 ± 337.0 | 3168.4 ± 278.2 |
| M3 | IR | 3776.0 ± 351.9 | 7559.7 ± 137.5 | 8245.8 ± 352.1 | 9066.0 ± 973.8 | 9328.0 ± 624.6 |
| M4 | IR | 0 | 0 | 0 | 0 | 0 |
| M5 | ER | 191.0 ± 94.1 | 192.4 ± 28.2 | 185.5 ± 23.4 | 190.7 ± 33.0 | 182.9 ± 21.4 |
| M6 | ER | 91.0 ± 61.2 | 53.6 ± 11.2 | 83.8 ± 13.4 | 90.0 | 48.54 ± 23.6 |
| M7 | ER | 1473.0 ± 47.3 | 2601.8 ± 127.6 | 2885.7 ± 68.0 | 2924.5 ± 86.9 | 3288.4 ± 169.8 |
| M8 | ER | 85.0 ± 7.0 | 102.8 ± 12.2 | 66.1 ± 17.1 | 100.0 | 89.7 ± 5.0 |
| M9 | ER | 0 | 40.1 ± 6.0 | 0 | 0 | 104.6 ± 24.4 |
| M10 | ER | 423.0 ± 55.8 | 896.4 ± 70.0 | 1082.7 ± 112.3 | 1121.2 ± 75.2 | 1179.9 ± 67.5 |

Example 1. Stable extended release (ER) formulation of metformin hydrochloride (HCl) is prepared by direct compression (Stable Formulation-1). Briefly, metformin HCl is mixed with polyethylene oxide 7 million molecular weight (POLYOX™ WSR 303 LEO) followed by lubrication with magnesium stearate and compression (Table 8). The compressed tablet met dissolution specification of U.S. Pharmacopeia (USP) and comparable to commercial ER product of metformin. The tablets packed in HDPE bottle were exposed to 40° C./75% RH and 25° C./60% RH for 12 and 18 months, respectively and monitored for NDMA impurity. NDMA level of 92.2±4.6 ng and 42.8 ng was observed after 12- and 18-months storage at 40° C./75% RH and 25° C./60% RH, respectively. Thus, formulation was stable against the formation of NDMA impurity.

TABLE 8
Stable Formulation-1 composition of metformin HCl ER tablets.

| | Amount (mg) | | | |
|---|---|---|---|---|
| Metformin HCl | 500 | 750 | 850 | 1000 |
| POLYOX ™ (WSR 303 LEO NF) | 322 | 483 | 547.4 | 644 |
| (Containing 0.010-0.100% | | | | |
| Butylated Hydroxytoluene) | | | | |
| Magnesium Stearate | 8 | 12 | 13.6 | 16 |
| Total | 830 | 1245 | 1411 | 1660 |

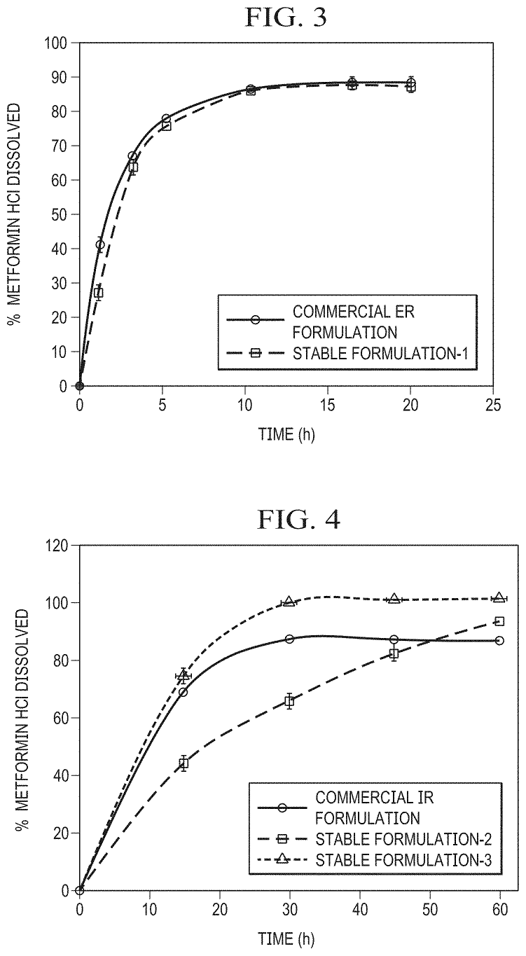

FIG. 3 illustrates comparative dissolution profiles of commercial and stable extended release (ER) formulations of metformin hydrochloride.

Example 2 and Example 3. Stable immediate release (IR) formulation of metformin HCl is prepared by direct compression. Briefly, metformin is mixed with polyethylene oxide 100,000 molecular weight (POLYOX™ WSR N10) and magnesium aluminum silicate (Stable Formulation-2) or dicalcium phosphate (Stable Formulation-3) followed by lubrication with magnesium stearate and compression (Table 9 and Table 10). Magnesium aluminum silicate is used as diluent and polyethylene oxide is a dry binder. The compressed tablet met dissolution specification of USP. The tablets packed in HDPE bottle were exposed to 40° C./75% RH and 25° C./60% RH for 12 and 18 months, respectively, and monitored for NDMA impurity. NDMA level in stable formulations based on POLYOX™ and dicalcium phosphate after 12 months exposure to 40° C./75% RH was 38.5±5.5 ng and 0 ng, respectively. Similarly, NDMA content 80.4 and 56.2 ng in POLYOXTM and dicalcium phosphate based formulations were observed after 18 months exposure to 25° C./60% RH, respectively (Table 11 and Table 12). Thus, the formations were stable against the formation of NDMA impurity.

TABLE 9
Stable Formulation-2 composition of metformin HCl IR tablets.

| | Amount (mg) | | | |
|---|---|---|---|---|
| Metformin HCl | 500 | 750 | 850 | 1000 |
| POLYOX ™ (WSR N10 LEO NF) | 100 | 150 | 170 | 200 |
| (Containing 0.005-0.025% | | | | |
| Butylated Hydroxytoluene) | | | | |
| Magnesium Aluminum Silicate | 43 | 64.5 | 73.1 | 86 |
| Magnesium Stearate | 7 | 10.2 | 11.9 | 14 |
| Total | 650 | 974.7 | 1105 | 1300 |

TABLE 10
Stable Formulation-3 composition of metformin HCl IR tablets.

| | Amount (mg) | | | |
|---|---|---|---|---|
| Metformin HCl | 500 | 750 | 850 | 1000 |
| Dicalcium Phosphate | 100 | 150 | 170 | 200 |
| Magnesium Aluminum Silicate | 43 | 64.5 | 73.1 | 86 |
| Magnesium Stearate | 7 | 10.2 | 11.9 | 14 |
| Total | 650 | 974.7 | 1105 | 1300 |

FIG. 4 illustrates comparative dissolution profiles of commercial and stable immediate release (IR) formulations of metformin hydrochloride.

Summary of results for selected IR and ER metformin compositions. Table 11 and Table 12, shown below, illustrate NDMA level in ng/500 mg metformin tablets at 40° C./75% RH and 25° C./60% RH respectively.

TABLE 11
Evaluation of stable metformin formulations stored at 40° C./75% RH.
40° C./75% RH
NDMA in ng/500 mg Metformin

| | Initial | 2 W | 1 M | 2 M | 3 M | 6 M | 9 M | 12 M |
|---|---|---|---|---|---|---|---|---|
| ER | 0 | 0 | 0 | 0 | 0 | 0 | 0 | 0 |
| Formulation-1 | | | | | | | | |
| IR | 0 | 0 | 0 | 0 | 0 | 0 | 68.3 | 92.2 |
| Formulation-2 | | | | | | | | |
| IR | 0 | 0 | 0 | 0 | 0 | 0 | 0 | 38.5 |
| Formulation-3 | | | | | | | | |

TABLE 12
Evaluation of stable metformin formulations stored at 25° C./60% RH.
25° C./60% RH
NDMA in ng/500 mg Metformin

| | Initial | 18 M |
|---|---|---|
| ER Formulation-1 | 0 | 42.8 |
| IR Formulation-2 | 0 | 80.4 |
| IR Formulation-3 | 0 | 56.2 |

These new formulations have been shown to prevent NDMA formation. This is advantageous as NDMA is a known carcinogen, causing metformin and other drugs, to be recalled in several countries. With the concern that metformin can form NDMA if stored for a long period of time in heat and humidity, it is advantageous to manufacture metformin such that it is able to prevent NDMA formation. The compositions above do not form NDMA or level is below FDA limit, even if the formulation is stored at 40° C. and 75% RH in a HDPE bottle. Currently, the FDA allows up to 96 ng/day of NDMA formation in metformin. If the amount is greater than that, the products are recalled from markets as they are considered harmful. The above formulations meet, or fall below, the 96 ng/day requirement imposed by the FDA.

In view of the aforementioned, the present disclosure generally relates to pharmaceutical compositions and manufacturing, and in particular, but not by way of limitation on new pharmaceutical compositions of primary, secondary, and/or tertiary amines group containing drugs with improved stability. The present disclosure utilizes the new pharmaceutical compositions for the formulation, preparation, or manufacturing of immediate release, extended release or controlled release tablets, capsules, pills, granules, pellets, solutions, suspensions, emulsions, and semi-solid formulations. These pharmaceutical compositions are stable and reduce the formation of nitroso impurities in the pharmaceutical compositions. In some embodiments, the excipients of the present disclosure can be added to existing commercial compositions to form drugs with improved stability.

In a particular aspect, the present disclosure pertains to a stable pharmaceutical composition of a primary, secondary, and/or tertiary amino group containing drugs. In general, the compositions include, without limitation: (a) a primary, secondary, and/or tertiary amino group containing drug(s); (b) a protective pharmaceutical excipient(s); and optionally (c) a pharmaceutically acceptable processing aid(s), such as, for example, a flow promotor, a solvent, a bulking agent, and the like. In some embodiments, the compositions of the present disclosure: (i) contain at least one drug of primary, secondary, and/or tertiary amino group containing drugs and at least one protective pharmaceutical excipient; and (ii) has a mass ratio of drug to protective pharmaceutical excipient(s) ranging from 0.1 to 99 to 99 to 1% w/w.

In another aspect, the present disclosure provides a stable formulation of an immediate release, extended release or controlled release tablet, delayed release or enteric release, delayed extended release or enteric extended release, capsule, pills, granules, pellets, solution, suspension, emulsion, and/or semi-solid dosage forms. Additionally, the present disclosure provides a stable formulation to protect or reduce the formation of nitroso impurities when exposed to room temperature, in-use and accelerated temperatures and humidity conditions.

In another embodiment, the present disclosure pertains to a stable formulation of primary, secondary, and/or tertiary amino group containing composition that prevents or reduces formation of a nitroso impurity. In some embodiments, the composition includes a drug having a primary, secondary, and/or tertiary amino group and a protective pharmaceutical excipient(s). In some embodiments, a weight ratio of the drug to the protective pharmaceutical excipient(s) ranges from 1 to 99 to 99 to 1% w/w.

In some embodiments, the drug including the primary, secondary, and/or tertiary amino group includes, without limitation, metformin, varenicline, quinapril, nizatidine, glipizide, glyburide, glimepiride, pioglitazone, rosiglitazone, repaglinide, alogliptin, linagliptin, sitagliptin, saxagliptin, candesartan, irbesartan, losartan, Olmesartan, valsartan, azilsartan, hydrochlorothiazide, amlodipine, nebivolol, sacubitril, aliskiren, ranitidine, cimetidine, omeprazole, rifampin, rifapentine, and their salts that are prone to formation of nitroso impurities.

In some embodiments, the composition is stable against the formation of a nitroso impurity. In some embodiments, the composition reduces formation of the nitroso impurity to an acceptable level. In some embodiments, the acceptable level nitroso impurities is at or below 26.5 to 96 ng/day. In some embodiments, the composition is stable against the formation of the nitroso impurity or reduces formation of the nitroso impurity when the composition is exposed to 25 ° C./75% RH or 40° C./75% RH. In some embodiments, the composition has a form that includes, without limitation, immediate release, extended release, delayed release, delayed extended release, controlled release, a tablet, a capsule, a pill, a granule, a pellet, a solution, a suspension, an emulsion, a semi-solid, and combinations thereof.

In some embodiments, the protective pharmaceutical excipient includes a cyclodextrin compound. In some embodiments, the cyclodextrin compound is present in the composition in a range from 1 to 95% w/w. In some embodiments, the cyclodextrin compound includes, without limitation, alpha-cyclodextrin, beta-cyclodextrin, gamma-cyclodextrin, randomly methylated beta-cyclodxetrin, hydropropyl beta-cyclodextrin, hydropropyl gamma-cyclodextrin, sulfobutyl ether beta-cyclodextrin, and combinations thereof.

In some embodiments, the protective pharmaceutical excipient includes a polydimethylsiloxane compound. In some embodiments, the polydimethylsiloxane compound is present in the composition in a ranged from 1 to 95% w/w. In some embodiments, the polydimethylsiloxane compound includes, without limitation, dimethicone, cyclomethicone, silica dimethyl silylate, simethicone, and combinations thereof.

In some embodiments, the protective pharmaceutical excipient includes a polyhydric alcohol. In some embodiments, the polyhydric alcohol is present in the composition in a range from 1 to 95% w/w. In some embodiments, the polyhydric alcohol includes, without limitation, glycerin, propylene glycol, butylene glycol, propylene carbonate, monothioglycerol, polyethylene glycol (molecular weight of less than 1,000), and combinations thereof.

In some embodiments, the protective pharmaceutical excipient includes a cyclodextrin compound. In some embodiments, the cyclodextrin compound is present in the composition in a range from 1 to 95% w/w. In some embodiments, the cyclodextrin compound includes, without limitation, alpha-cyclodextrin, beta-cyclodextrin, gamma-cyclodextrin, randomly methylated beta-cyclodxetrin, hydropropyl beta-cyclodextrin, hydropropyl gamma-cyclodextrin, sulfobutyl ether beta-cyclodextrin, and combinations thereof.

In some embodiments, the protective pharmaceutical excipient includes a polydimethylsiloxane compound. In some embodiments, the polydimethylsiloxane compound is present in the composition in a ranged from 1 to 95% w/w. In some embodiments, the polydimethylsiloxane compound includes, without limitation, dimethicone, cyclomethicone, silica dimethyl silylate, simethicone, and combinations thereof.

In some embodiments, the protective pharmaceutical excipient includes a polyhydric alcohol. In some embodiments, the polyhydric alcohol is present in the composition in a range from 1 to 95% w/w. In some embodiments, the polyhydric alcohol includes, without limitation, glycerin, propylene glycol, butylene glycol, propylene carbonate, monothioglycerol, polyethylene glycol (molecular weight of less than 1,000), and combinations thereof.

In some embodiments, the protective pharmaceutical excipient includes a calcium salt. In some embodiments, the calcium salt is present in the composition in a range from 1 to 95% w/w. In some embodiments, the calcium salt includes, without limitation, calcium carbonate, dicalcium phosphate, tricalcium phosphate, calcium sulfate, calcium citrate, calcium pyrophosphate, calcium silicate, calcium trisilicate, calcium stearate, sodium calcium aluminosilicate, and combinations thereof.

In some embodiments, the protective pharmaceutical excipient includes a magnesium salt. In some embodiments, the magnesium salt is present in the composition in a range from 1 to 95% w/w. In some embodiments, the magnesium salt includes, without limitation, magnesium aluminum silicate, magnesium aluminometasilicate, magnesium carbonate, magnesium oxide, magnesium silicate, magnesium stearate, magnesium sulfate, magnesium trisilicate, and combinations thereof.

In some embodiments, the protective pharmaceutical excipient includes at least one of a sodium, potassium, or aluminum salt. In some embodiments, the at least one of a sodium, potassium, or aluminum salt is present in the composition in a range from 1 to 95% w/w. In some embodiments, the at least one of a of sodium, potassium, and aluminum salt includes, without limitation, sodium silicate, potassium silicate, sodium aluminosilicate, and combinations thereof.

In some embodiments, the protective pharmaceutical excipient includes, without limitation, kaolin, bentonite, and silicon dioxide. In some embodiments, the protective pharmaceutical excipient includes, without limitation, kaolin, bentonite, and silicon dioxide and is present in the composition in a range from 1 to 95% w/w.

In some embodiments, the protective pharmaceutical excipient is a long carbon chain acid. In some embodiments, the long carbon chain acid is saturated or unsaturated with carbon length varied from 4 to 26. In some embodiments, the long carbon chain acid is present in the composition in a range from 1 to 95% w/w. In some embodiments, the long carbon chain acid includes, without limitation, lauric acid, myristic acids, palmitic acids, stearic acid, adipic acid, lipoic acid, omega-3 fatty acids, and combinations thereof.

In some embodiments, protective pharmaceutical excipient includes a long chain carbon alcohol. In some embodiments, the long chain carbon alcohol is present in the composition in a range from 1 to 95% w/w. In some embodiments, the long chain carbon alcohol is saturated or unsaturated, and wherein carbon length varies from 4 to 26. In some embodiments, the long chain carbon alcohol includes, without limitation, cetyl alcohol, cetostearyl alcohol, stearyl alcohol, tocopherol, isobutyl alcohol, myristyl alcohol, octyldodecanol, oleyl alcohol, lanolin alcohols, cholesterol, and combinations thereof.

In some embodiments, the protective pharmaceutical excipient includes at least one of polyethylene glycol or polyethylene oxide. In some embodiments, the at least one of polyethylene glycol or polyethylene oxide is present in the composition in a range from 1 to 95% w/w. In some embodiments, the at least one of polyethylene glycol or polyethylene oxide has a molecular weight that varies from 100 to 10,000,000.

In some embodiments, the protective pharmaceutical excipient includes at least one of cellulose acetate, cellulate butyrate, ethyl cellulose, or cellulose acetate. In some embodiments, the at least one of cellulose acetate, cellulate butyrate, ethyl cellulose, or cellulose acetate is present in the composition in a range from 1 to 95% w/w.

In some embodiments, the protective pharmaceutical excipient includes at least one of polydecene or hydrogenated polydecene. In some embodiments, the at least one of polydecene or hydrogenated polydecene is present in the composition in a ranged from 1 to 95% w/w.

In some embodiments, the protective pharmaceutical excipient includes a polymer that includes, without limitation, acrylic acid (CARBOPOL®), amino methacrylate copolymer, ammonio methacrylate copolymer, ethyl acrylate and methyl methacrylate copolymer, methacrylic acid and ethyl acrylate copolymer, methacrylic acid and methyl methacrylate copolymer, and combinations thereof. In some embodiments, the polymer is present in the composition in a range from 1 to 95% w/w.

In some embodiments, the protectant includes an antioxidant including, without limitation, butylated hydroxy anisole (BHA), butylated hydroxy toluene (BHT), sodium/potassium metabisulfites, sodium/potassium sulfite, cysteine, methionine, sodium or calcium ascorbate, ascorbic acid, fatty acid esters of ascorbic acid, tocopherols, alpha, gamma or delta tocopherol and its esters, 4-hydroxyresorcinol, erythorbic acid, sodium erythorbate, propylgallate, octyl gallate, tertiary butyl hydroquinone, and combinations thereof. In some embodiments, the antioxidant is present in the composition in a range from 0.001 to 5%.

In some embodiments, the protectant includes a chelating agent including, without limitation, ethylenediaminetetraacetic acid, edetate sodium, edetate disodium, edetate calcium disodium, edetate tripotassium, edetate dipotassium, and combination thereof. In some embodiments, the antioxidant is present in the composition in a range from 0.001 to 5%.

In an additional embodiment, the present disclosure pertains to a method of making a stabilized formulation of a primary, secondary, and/or tertiary amino group containing composition that prevents or reduces formation of a nitroso impurity. In some embodiments, the method includes adding a pharmaceutical excipient(s) to a drug(s) having a primary, secondary, and/or tertiary amino group. In some embodiments, a weight ratio of the drug(s) to the protective pharmaceutical excipient(s) ranges from 1 to 99 to 99 to 1% w/w. In some embodiments, the pharmaceutical excipient(s) is a protective pharmaceutical excipient. In some embodiments, the pharmaceutical excipient is processed with an organic solvent.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

What is claimed is:

1. A stable formulation of a primary, secondary, and/or tertiary amino group containing composition that prevents or reduces formation of a nitroso impurity until the end of stated expiration or longer, the composition comprising:
metformin; and
at least one pharmaceutical excipient, wherein the at least one pharmaceutical excipient comprises:
a magnesium salt selected from the group consisting of magnesium aluminum silicate, magnesium aluminometasilicate, magnesium carbonate, magnesium oxide, magnesium silicate, magnesium stearate, magnesium sulfate, magnesium trisilicate, and combinations thereof; and
at least one of a calcium salt, calcium carbonate, dicalcium phosphate, tricalcium phosphate, calcium sulfate, calcium silicate, calcium trisilicate, calcium stearate, sodium calcium aluminosilicate, polyethylene glycol, or polyethylene oxide;
wherein a weight ratio of the metformin to the at least one pharmaceutical excipient ranges from 1 to 99 to 99 to 1% w/w; and
wherein the at least one pharmaceutical excipient is a protective pharmaceutical ingredient.

2. The composition of claims 1, wherein the composition further comprises at least one other drug.

3. The composition of claims 2, wherein the at least one other drug comprises a tertiary or quaternary amine or non-amine groups in its structure.

4. The composition of claims 2, wherein the at least one other drug is selected from the group consisting of dapagliflozin, empagliflozin, ertugliflozin, and canagliflozin.

5. The composition of claims 1, wherein the nitroso impurity is selected from the group consisting of N-Nitrosovarenicline, N-nitrosoquinapril, N-nitroso sitagliptin, N-nitrosodimethylamine, N-nitrosodiethylamine, N-nitroso-N-methyl-4-aminobutanoic acid, N-nitrosoisopropylethyl amine, N-nitrosodiisopropylamine, N-nitrosodibutylamine, N-nitroso-varenicline, N-nitroso-irbesartan, 1-methyl-4-nitrosopiperazine, N-nitrosoquinapril, 1-cyclopentyl-4-nitrosopiperazine and N-nitrosomethylphenylamine as nitroso impurities, nitroso group containing molecules, and combinations thereof.

6. The composition of claims 1, wherein the composition is stable against formation of the nitroso impurity when the composition packed in high-density polyethylene (HDPE) bottle, blister pack or any of other finished container is exposed to 25° C./60% relative humidity (RH) for 18 months or longer or 40° C./75% RH for six months or longer.

7. The composition of claims 1, wherein the composition reduces formation of the nitroso impurity to an acceptable level, and wherein the acceptable level is at or below 26.5 to 96 ng throughout the shelf-life.

8. The composition of claims 1, wherein the composition is stable against formation of the nitroso impurity, or reduces formation of the nitroso impurity, when the composition is exposed to 25° C./60% relative humidity (RH) or 40° C./75% RH, or during in-use condition (30° C./75% RH).

9. The composition of claims 1, wherein the composition has a form selected from the group consisting of immediate release, extended release, delayed release, delayed extended release, controlled release, a tablet, a capsule, a pill, a granule, a pellet, a solution, a suspension, an emulsion, a semi-solid, and combinations thereof.

10. The composition of claims 1, wherein the at least one pharmaceutical excipient comprises a cyclodextrin compound present in the composition in a range from 1 to 95% w/w, and wherein the cyclodextrin compound is selected from the group consisting of alpha-cyclodextrin, beta-cyclodextrin, gamma-cyclodextrin, randomly methylated beta-cyclodxetrin, hydropropyl beta-cyclodextrin, hydropropyl gamma-cyclodextrin, sulfobutyl ether beta-cyclodextrin, and combinations thereof.

11. The composition of claims 1, wherein the at least one pharmaceutical excipient comprises a polydimethylsiloxane compound present in the composition in a range from 1 to 95% w/w, and wherein the polydimethylsiloxane compound is selected from the group consisting of dimethicone, cyclomethicone, silica dimethyl silylate, simethicone, and combinations thereof.

12. The composition of claims 1, wherein the at least one pharmaceutical excipient comprises a polyhydric alcohol selected from the group consisting of glycerin, propylene glycol, butylene glycol, propylene carbonate, monothioglycerol, polyethylene glycol (molecular weight of less than 1,000), and combinations thereof, and wherein the polyhydric alcohol is present in the composition in a range from 1 to 95% w/w.

13. The composition of claims 1, wherein the at least one pharmaceutical excipient comprises a calcium salt present in the composition in a range from 1 to 95% w/w, and wherein the calcium salt is selected from the group consisting of calcium carbonate, dicalcium phosphate, tricalcium phosphate, calcium sulfate, calcium citrate, calcium pyrophosphate, calcium silicate, calcium trisilicate, calcium stearate, sodium calcium aluminosilicate, and combinations thereof.

14. The composition of claims 1, wherein the magnesium salt is present in the composition in a range from 1 to 95% w/w.

15. The composition of claims 1, wherein the at least one pharmaceutical excipient comprises at least one of a sodium, potassium, or aluminum salt present in the composition in a range from 1 to 95% w/w, and wherein the at least one of a of sodium, potassium, and aluminum salt is selected from the group consisting of sodium silicate, potassium silicate, sodium aluminosilicate, and combinations thereof.

16. The composition of claims 1, wherein the at least one pharmaceutical excipient comprises at least one of kaolin, bentonite, or silicon dioxide present in the composition in a range from 1 to 95% w/w.

17. The composition of claims 1, wherein the at least one pharmaceutical excipient comprises a long carbon chain acid, saturated or unsaturated, with carbon length varied from 4 to 26, and wherein the long carbon chain acid is selected from the group consisting of lauric acid, myristic acids, palmitic acids, stearic acid, adipic acid, lipoic acid, omega-3 fatty acids, and combinations thereof.

18. The composition of claims 1, wherein the at least one pharmaceutical excipient comprises a long chain carbon alcohol present in the composition in a range from 1 to 95% w/w, and wherein the long chain carbon alcohol is selected from the group consisting of cetyl alcohol, cetostearyl alcohol, stearyl alcohol, tocopherol, isobutyl alcohol, myristyl alcohol, octyldodecanol, oleyl alcohol, lanolin alcohols, cholesterol, and combinations thereof.

19. The composition of claims 1, wherein the at least one of dicalcium phosphate, polyethylene glycol, or polyethylene oxide comprises at least one of polyethylene glycol or polyethylene oxide with a molecular weight that varies from 1,000 to 10,000,000.

20. The composition of claims 1, wherein the at least one pharmaceutical excipient comprises at least one of cellulose acetate, cellulate butyrate, ethyl cellulose, or cellulose acetate present in the composition in a range from 1 to 95% w/w.

21. The composition of claims 1, wherein the at least one pharmaceutical excipient comprises at least one of polydecene or hydrogenated polydecene present in the composition in a range from 1 to 95% w/w.

22. The composition of claims 1, wherein the pharmaceutical excipient comprises a polymer selected from the group consisting of acrylic acid, amino methacrylate copolymer, ammonio methacrylate copolymer, ethyl acrylate and methyl methacrylate copolymer, methacrylic acid and ethyl acrylate copolymer, methacrylic acid and methyl methacrylate copolymer, and combinations thereof, and wherein the polymer is present in the composition in a range from 1 to 95% w/w.

23. The composition of claim 1, wherein the composition comprises an antioxidant.

24. The composition of claim 23, wherein the antioxidant is selected from the group consisting of butylated hydroxy anisole, butylated hydroxy toluene, sodium/potassium metabisulfites, sodium/potassium sulfite, cysteine, methionine, sodium or calcium ascorbate, fatty acid esters of ascorbic acid, tocopherols, alpha, gamma or delta tocopherol and its esters, 4-hydroxyresorcinol, erythorbic acid, sodium erythorbate, propyl gallate, octyl gallate, tertiary butyl hydroquinone, and combinations thereof, and wherein the antioxidant is present in the composition in a range from 0.001 to 5% w/w.

25. The composition of claim 1, wherein the composition comprises a chelating agent.

26. The composition of claim 1, wherein the chelating agent is selected from the group consisting of ethylenediaminetetraacetic acid, edetate sodium, edetate disodium, edetate calcium disodium, edetate tripotassium, edetate dipotassium, and combination thereof, and wherein the chelating agent is present in the composition in a range from 0.001 to 5% w/w.

27. The composition of claim 1, wherein the magnesium salt comprises magnesium stearate, and wherein the at least one of a calcium salt, calcium carbonate, dicalcium phosphate, tricalcium phosphate, calcium sulfate, calcium silicate, calcium trisilicate, calcium stearate, sodium calcium aluminosilicate, polyethylene glycol or polyethylene oxide comprises polyethylene oxide.

28. A stable formulation of a primary, secondary, and/or tertiary amino group containing composition that prevents or reduces formation of a nitroso impurity until the end of stated expiration or longer, the composition comprising:
metformin; and
at least one pharmaceutical excipient, wherein the at least one pharmaceutical excipient comprises:
magnesium stearate; and
at least one of dicalcium phosphate, polyethylene glycol, or polyethylene oxide;
wherein a weight ratio of the metformin to the at least one pharmaceutical excipient ranges from 1 to 99 to 99 to 1% w/w; and
wherein the at least one pharmaceutical excipient is a protective pharmaceutical ingredient.

29. A method of making a stabilized formulation of a primary, secondary, and/or tertiary amino group containing composition that prevents or reduces formation of a nitroso impurity, the method comprising:
adding a pharmaceutical excipient to metformin;
wherein the pharmaceutical excipient comprises:
a magnesium salt selected from the group consisting of magnesium aluminum silicate, magnesium aluminometasilicate, magnesium carbonate, magnesium oxide, magnesium silicate, magnesium stearate, magnesium sulfate, magnesium trisilicate, and combinations thereof; and
at least one of calcium carbonate, dicalcium phosphate, tricalcium phosphate, calcium sulfate, calcium silicate, calcium trisilicate, calcium stearate, sodium calcium aluminosilicate, polyethylene glycol, or polyethylene oxide;
wherein a weight ratio of the metformin to the pharmaceutical excipient ranges from 1 to 99 to 99 to 1% w/w; and
wherein the pharmaceutical excipient is a protective pharmaceutical agent.

30. The method of claim 29, wherein the magnesium salt comprises magnesium stearate, and wherein the at least one of dicalcium phosphate, polyethylene glycol, or polyethylene oxide comprises polyethylene oxide.
